SEQUENCE LISTING (3 sequences)

SEQ ID NO: 1 - protein, 30 aa, Homo sapiens
HAEGTFTSDVSSYLEGQAAKEFIAWLVKGR

SEQ ID NO: 2 - protein, 29 aa, Homo sapiens
HAEGTFTSDVSSYLEGQAAKEFIAWLVKG

SEQ ID NO: 3 - protein, 31 aa, Homo sapiens
HAEGTFTSDVSSYLEGQAAKEFIAWLVKGRG

(19) United States
(12) Patent Application Publication
(10) Pub. No.: US 2023/0201311 A1
(43) Pub. Date: Jun. 29, 2023

(54) COMPOSITIONS AND METHODS FOR TREATING NON-ALCOHOLIC STEATOHEPATITIS (NASH)

(71) Applicant: SHANGHAI BENEMAE PHARMACEUTICAL CORPORATION, Shanghai (CN)

(72) Inventors: Zhiqiang DU, Shanghai (CN); Xiankang FANG, Shanghai (CN); Qingqin LAI, Shanghai (CN); Yan QIU, Shanghai (CN); Peng WU, Shanghai (CN)

(73) Assignee: SHANGHAI BENEMAE PHARMACEUTICAL CORPORATION, Shanghai (CN)

(21) Appl. No.: 17/813,278

(22) Filed: Jul. 18, 2022

Related U.S. Application Data
(63) Continuation of application No. PCT/CN2021/072204, filed on Jan. 15, 2021.
Application No. PCT/CN2021/072204 is a continuation of application No. PCT/CN2020/072555, filed on Jan. 16, 2020.

(51) Int. Cl. A61K 38/26; A61P 1/16

(52) U.S. Cl. CPC A61K 38/26; A61P 1/16

(57) ABSTRACT

Disclosed herein are compositions comprising GLP-1 and/or GLP-1 analog(s) for treating or preventing non-alcoholic steatohepatitis (NASH).

### PRIORITY CLAIM

This application is a continuation of International Patent Application No. PCT/CN2021/072204, filed Jan. 15, 2021, which is a continuation of International Patent Application No. PCT/CN2020/072555, filed Jan. 16, 2020, both of which are incorporated by reference herein in their entirety.

### SEQUENCE LISTING

This application contains a Sequence Listing, which was submitted in XML format via Patent Center, and is hereby incorporated by reference in its entirety. The XML copy, created on Mar. 16, 2023, is named 057783.8016.US01.xml and is 4,000 bytes in size.

### BACKGROUND

Non-alcoholic fatty liver disease (NAFLD) is a condition caused by fat buildup in the liver such that the liver functions are impacted. Non-alcoholic steatohepatitis (NASH) is an advanced stage of NAFLD, in which the liver suffers inflammation and damage. Severe NASH can lead to liver scarring, and potentially life-threatening cirrhosis or liver cancer. NASH may have few or no symptoms but are associated with certain health issues such as obesity, metabolic syndrome, diabetes, cardiovascular morbidity and mortality, etc. Other than weight control and restricted diet, currently there is no standard treatment for NASH.

GLP-1 is an insulinotropic peptide acting on GLP-1 receptor expressed particularly on pancreatic insulin-secreting 0 cells and on neurons of the brain. The native form of GLP-1 is secreted by intestinal L-cells after a meal, and is a strong peptide stimulator of insulin secretion. GLP-1 is a potential therapy for type 2 diabetes. Hoist, Physiol. Rev. 87: 1409-1439 (2007). GLP-1 has two active forms in human body, GLP-1 (7-36) with a C-terminal amide and GLP-1 (7-37) with a C-terminal free carboxyl group. Upon entry into circulation, GLP-1 is rapidly degraded by dipeptidyl peptidase-4 (DPP4), resulting in a short half-life of about 2 minutes. Kieffer et al., Endocrinology 136: 3585-3596 (1995). Besides its effect on controlling blood sugar, GLP-1 and its analogs were found to have effects on inducing body weight loss, slowing stomach emptying, and increasing satiety. Monami et al., Exp. Diabetes Res. 2012: 672658 (2012). FDA approved Saxenda (liraglutide 3 mg) in December 2014, which is the first NDA application for GLP-1 analogs, and which is a long-term weight management supplemented with low-calorie diet and increase of physical activity for obesity adults having at least one disease or condition associated with overweight, such as type 2 diabetes. Continuing efforts are made to further improve the efficacy of GLP-1 analogs. This disclosure provides a treatment and prevention method for NASH using GLP-1 and/or GLP-1 analogs disclosed herein.

### SUMMARY

In one aspect, provided is a method of treating or preventing non-alcoholic steatohepatitis (NASH) in a subject. The method entails administering to a subject an effective amount of one or more first active ingredients selected from the group consisting of GLP-1 and GLP-1 analogs. In some embodiments, the subject has NASH or has an elevated risk of having NASH. In some embodiments, the GLP-1 analogs are selected from the group consisting of GLP-1 (7-37), GLP-1 (7-36), and GLP-1 (7-35). In some embodiments, the GLP-1 and/or GLP-1 analog(s) (e.g., GLP-1 (7-36) such as Beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as Beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight.

In some embodiments, the GLP-1 and/or GLP-1 analog(s) (e.g., GLP-1 (7-36) such as Beinaglutide) may be administered once a day, twice a day, three times a day, or four times a day. In some embodiments, the total daily dosing of the GLP-1 and/or GLP-1 analog(s) (e.g., GLP-1 (7-36) such as Beinaglutide) may be about 40 μg to about 14,000 μg, about 40 μg to about 13,500 μg, about 50 μg to about 14,000 μg, about 50 μg to about 13,500 μg, about 40 μg to about 12,030 μg, about 50 μg to about 12,040 μg, about 2,010 μg to about 14,000 μg, about 1,510 μg to about 13,500 μg, about 250 μg to about 6,000 μg, about 250 μg to about 5,700 μg, about 300 μg to about 6,000 μg, about 300 μg to about 5,700 μg, about 480 μg to about 700 μg, about 480 μg to about 600 μg, about 540 μg to about 700 μg, or about 540 μg to about 600 μg.

In another aspect, provided is a pharmaceutical composition comprising an effective amount of one or more first active ingredients selected from the group consisting of GLP-1 and GLP-1 analogs for treating or preventing NASH in a subject. In some embodiments, the subject has NASH or has at an elevated risk of having NASH. In some embodiments, the GLP-1 analog is selected from the group consisting of GLP-1 (7-37), GLP-1 (7-36), and GLP-1 (7-35). In some embodiments, the pharmaceutical composition comprising the GLP-1 and/or GLP-1 analog(s) (e.g., GLP-1 (7-36) such as Beinaglutide) at a concentration of 2 mg/mL. In some embodiments, the pharmaceutical composition comprising the GLP-1 and/or GLP-1 analog(s) (e.g., GLP-1 (7-36) such as Beinaglutide) is preloaded into an administration device (e.g., an injector pen, a pump).

In another aspect, provided is a kit for treating or preventing NASH in a subject, the kit comprising a pharmaceutical composition comprising an effective amount of one or more first active ingredients selected from the group consisting of GLP-1 and GLP-1 analogs (e.g., GLP-1 (7-36) such as Beinaglutide) as disclosed herein.

### BRIEF DESCRIPTION OF THE DRAWINGS

This application contains at least one drawing executed in color. Copies of this application with color drawing(s) will be provided by the Office upon request and payment of the necessary fees.

FIG. 1 shows the pharmacokinetics of Beinaglutide in C57BL/6J mice and HFD-NASH mice prepared as set forth in Example 1.

FIGS. 2A-2E show effects of Beinaglutide on steatosis in HFD-NASH mice prepared as set forth in Example 1. The animals of two groups were subcutaneously injected (3 mL/kg) three time per day for 4 weeks with vehicle (Vehicle) or Beinaglutide (2.4 mg/kg). FIG. 2A′ The comparison of the two groups in liver weight; FIG. 2B: The comparison of the two groups in liver triglyceride (TG) content; FIG. 2C: The comparison of the two groups in plasma alanine aminotransferase (ALT); FIG. 2D: The comparison of the two groups in plasma aspartate aminotransferase (AST); and FIG. 2E: The comparison of the two groups in lipid accumulation in representative hematoxylin and eosin (H&E)-stained liver sections, macrovesicular (shown by stars) and microvesicular (shown by arrows). The asterisks indicate a statistically significant difference according to Student's t-test (*P<0.05, **P<0.01).

FIGS. 3A-3E show effects of Beinaglutide on inflammation and fibrosis in HFD-NASH mice prepared as set forth in Example 1. The animals of two groups were subcutaneously injected (3 mL/kg) three time per day for 4 weeks with vehicle (Vehicle) or Beinaglutide (2.4 mg/kg). FIG. 3A: The comparison of the two groups in TNF-α; FIG. 3B: The comparison of the two groups in IL-6 levels; FIG. 3C: The comparison of the two groups in protein level of liver Col1a1; FIG. 3D: The comparison of the two groups in the expression of liver Col1a1 normalized to β-actin; FIG. 3E: The comparison of the two groups in hepatic fibrosis (black triangle, ▴) in representative liver sections stained by picrosirius red for collagen. The asterisks indicate a statistically significant difference according to Student's t-test (***P<0.001).

FIGS. 4A-4C show effects of Beinaglutide on body weight and insulin resistance in HFD-NASH mice prepared as set forth in Example 1. The animals of two groups were subcutaneously injected (3 mL/kg) three time per day for 4 weeks with vehicle (Vehicle) or Beinaglutide (2.4 mg/kg). FIG. 4A: The comparison of the two groups in body weight changes every day; FIG. 4B: the comparison of the two groups in oral glucose tolerance (OGTT) performed at day 30; FIG. 4C: The comparison of the two groups in HOMA-IR. The asterisks indicate a statistically significant difference according to Student's t-test (*P<0.05, **P<0.01).

FIG. 5 shows the animal experiment flow chart of the experiment for Example 2.

FIGS. 6A-6C: FIG. 6A shows the body weight, body weight change, liver weight and liver index of C57BL/6J and HFD-NASH mice after 17-week induction. ***P<0.001 vs C57BL/6J. FIG. 6B shows liver function and lipid metabolism analysis including serum ALT, serum AST, serum TBIL, serum TC, serum TG and serum LDL-c of C57BL/6J and HFD-NASH mice after 17-week induction. ***P<0.001 vs C57BL/6J. FIG. 6C shows liver TC and TG contents of C57BL/6J and HFD-NASH mice after 17-week induction. ***P<0.001 vs C57BL/6J.

FIGS. 7A-7C show liver tissue H&E staining of C57BL/6J and HFD-NASH mice after 17-week induction. FIG. 7A: Representative images of H&E staining of C57BL/6J and HFD-NASH mice. The area marked with “A” shows central veins, and the area marked with “B” shows the portal area. “$” shows microvesicular steatosis, “★” shows macrovesicular steatosis, and “→” shows inflammatory cells infiltration. FIG. 7B: NAS score of C57BL/6J and HFD-NASH mice. FIG. 7C: Fibrosis score of C57BL/6J and HFD-NASH mice. ***P<0.001 vs C57BL/6J.

FIGS. 8A-8C show the relative expression levels of COL1a1, TGFb1 and Acta2 to Gapdh of C57BL/6J and HFD-NASH mice after 17-week induction. *P<0.001 vs C57BL/6J.

FIGS. 9A-9B show Sirius red staining of C57BL/6J and HFD-NASH mice after 17-week induction. FIG. 9A: Representative images of Sirius red staining of C57BL/6J and HFD-NASH mice. The positive area is shown by an arrow. FIG. 9B: Statistics data of the positive area. ***P<0.001 vs C57BL/6J.

FIGS. 10A-10B show α-SMA IHC staining of C57BL/6J and HFD-NASH mice after 17-week induction. FIG. 10A: Representative images of IHC staining of C57BL/6J and HFD-NASH mice. The positive area is shown by an arrow. FIG. 10B: Statistics data of the positive area. ***P<0.001 vs C57BL/6J.

FIGS. 11A-11B show oil red staining of C57BL/6J and HFD-NASH mice after 17-week induction. FIG. 11A: Representative images of oil red staining of C57BL/6J and HFD-NASH mice. The positive area is shown by an arrow. FIG. 11B: Statistics data of the positive area. ***P<0.001 vs C57BL/6J.

FIG. 12 shows the body weight of various groups of the mice before Beinaglutide treatment started.

FIGS. 13A-13D show the body weight changes of various groups of the mice after Beinaglutide treatment. FIG. 13A: Body weight at the study end. FIG. 13B: Body weight changes vs control group (HFD-NASH disease model without any treatment) at the study end. FIG. 13C: Body weight changing curves. FIG. 13D: Percentage change of body weight from respective baselines. *P<0.05, **P<0.01, ***P<0.001 vs control group.

FIGS. 14A-14B show food consumption by various groups of the mice. FIG. 14A: Towards the end of study, the 8-hour average food consumption of each group in the last 3 measurements. FIG. 14B: During the dosing period, 8-hour average food consumption curves of each group. * P<0.05, *** P<0.001 vs control group.

FIGS. 15A-15B show the effects on liver weight and liver index, respectively after Beinaglutide treatment. *** P<0.001 vs control group.

FIGS. 16A-16B show the effects on ALT level before and after Beinaglutide treatment. FIG. 16A: Plasma ALT level before Beinaglutide treatment.

FIG. 168: Plasma ALT level changes within 9 weeks after Beinaglutide treatment. *P<0.05, **P<0.01 vs control group.

FIGS. 17A-17F show the effects on liver function (ALT, AST, TBIL, TG, TC, and LDL-c) after 11 weeks of Beinaglutide treatment. *P<0.05, ** P<0.01, * P<0.001 vs control group.

FIGS. 18A-18B show liver lipid contents (TC and TG) after 11 weeks of Beinaglutide treatment. *P<0.05, ***P<0.001 vs control group.

FIGS. 19A-19C show the expression levels of Col1a1, Tgfb1 and Acta2 genes relative to Gapdh after 11 weeks of Beinaglutide treatment. *P<0.05, **P<0.01 vs control group.

FIGS. 20A-20C show the severity of liver fibrosis after 11 weeks of Beinaglutide treatment. FIG. 20A: Representative images of H&E staining of control, Beinaglutide 0.6 mg/kg, Beinaglutide 1.2 mg/kg, and Beinaglutide 2.4 mg/kg mice. The area marked with “A” shows the central veins, and the area marked with B shows the portal area. “$” shows microvesicular steatosis, “★” shows macrovesicular steatosis, and “→” shows inflammatory cells infiltration. FIG. 20B: Fibrosis scores after H&E staining. *P<0.05 vs control group. FIG. 20C: Fibrosis severity grading after H&E staining.

FIGS. 21A-21C show the Sirius red staining results after 11 weeks of Beinaglutide treatment. FIG. 21A: Representative images of Sirius red staining of control, Beinaglutide 0.6 mg/kg, Beinaglutide 1.2 mg/kg, and Beinaglutide 2.4 mg/kg mice. The positive area is shown by an arrow. FIG. 21B: Positive area statistics after Sirius red staining. FIG. 21C: Fibrosis pattern after Sirius red staining.

FIGS. 22A-22B show the IHC staining results after 11 weeks of Beinaglutide treatment. FIG. 22A: Representative images of α-SMA IHC staining of control, Beinaglutide 0.6 mg/kg, Beinaglutide 1.2 mg/kg, and Beinaglutide 2.4 mg/kg. The positive area is shown by an arrow. FIG. 22B: Positive area statistics after IHC staining.

FIGS. 23A-23B show the H&E staining results after 11 weeks of Beinaglutide treatment. FIG. 23A: NAS scores. FIG. 23B: Macrosteatosis areas. **P<0.01 vs control group.

FIGS. 24A-24F show the evaluation of NASH severity after 11 weeks of Beinaglutide treatment.

### DETAILED DESCRIPTION

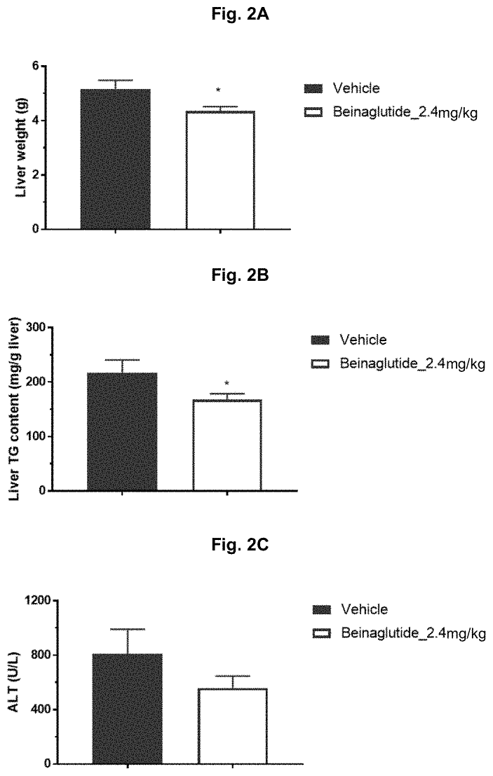

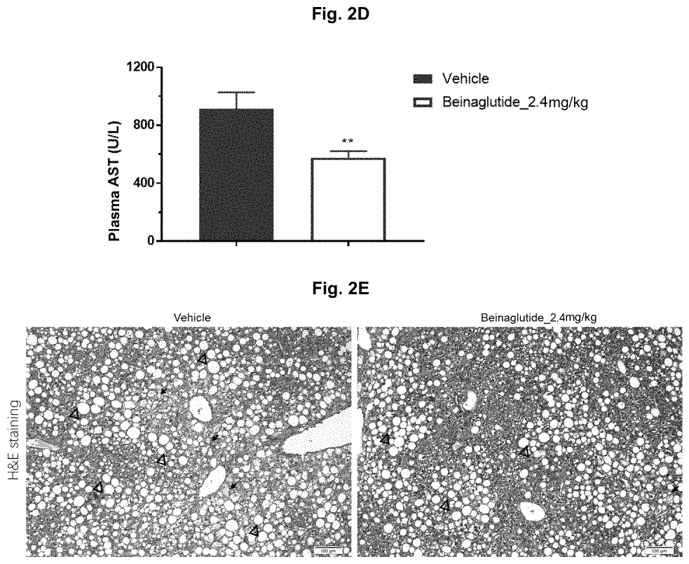

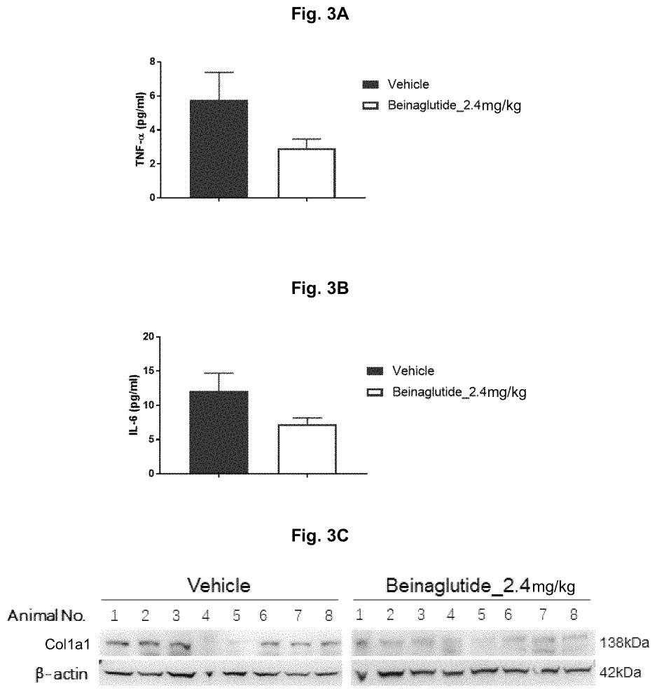

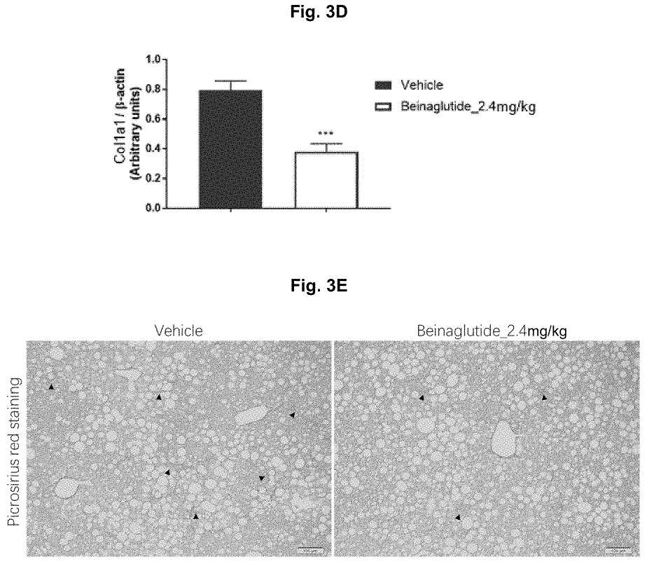

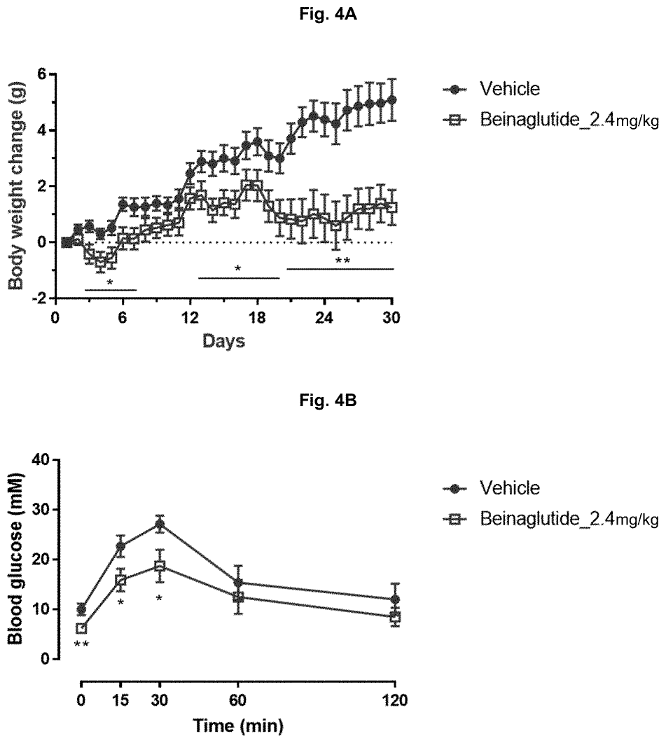

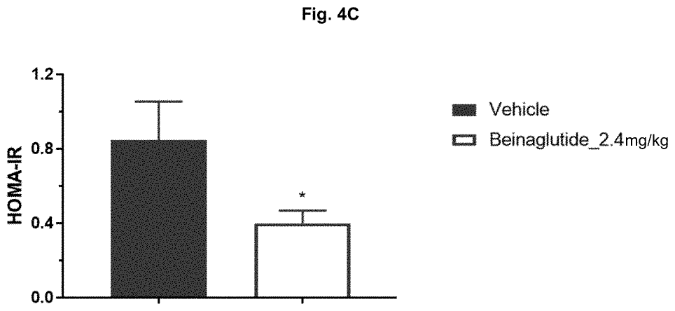

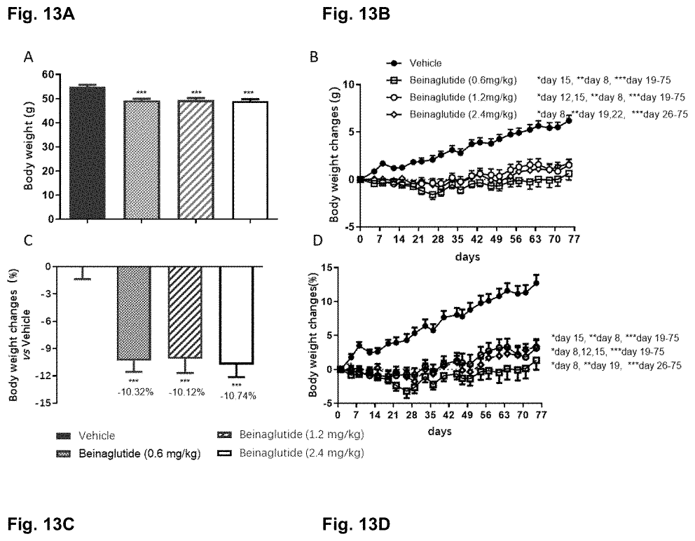

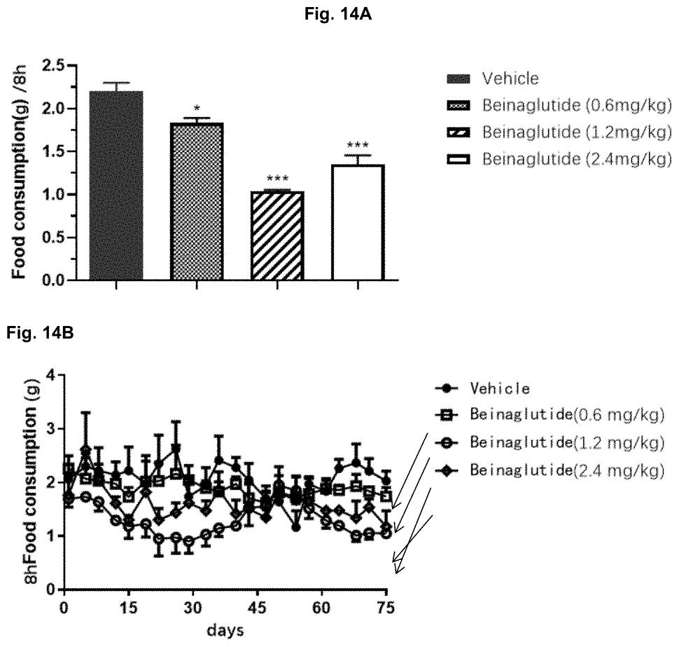

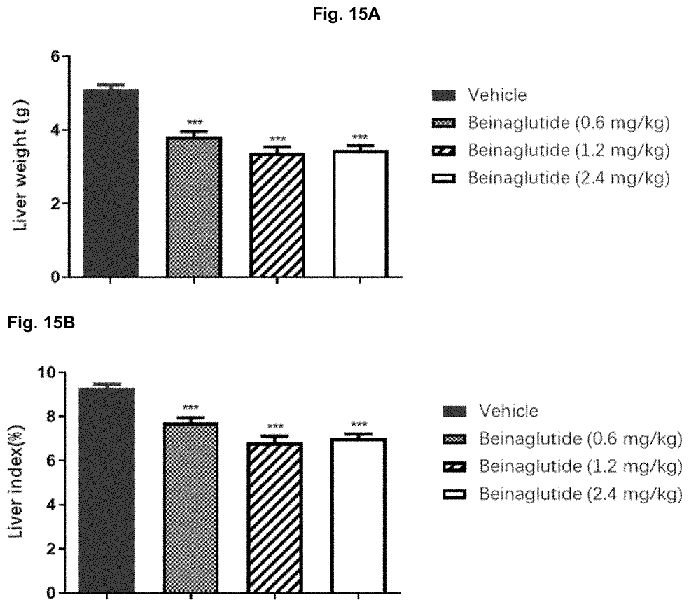

GLP-1 and GLP-1 analogs disclosed herein may be beneficial in treating NASH. For example, Beinaglutide treatment (2.4 mg/kg) showed significant improvement for NASH Indicators in HFD-NASH mice (Example 1): reduction of liver steatosis indicators (FIGS. 2A-2E); 2) improvement of inflammation and fibrosis indicators (FIGS. 3A-3E); and 3) reduction of weight (FIGS. 4A-4C). For example, liver steatosis indicators such as liver weight (FIG. 2A), liver triglyceride (TG) content (FIG. 2B), plasma alanine aminotransferase (ALT, FIG. 2C) and plasma aspartate aminotransferase (AST, FIG. 2D) were significantly reduced in HFD-NASH mice treated with Beinaglutide (2.4 mg/kg) compared with subjects treated with vehicle (the negative control). Beinaglutide treatment significantly reduced both macrovesicular (FIG. 2E, solid triangle) and microvesicular (FIG. 2E, arrow) in liver. Furthermore, markers involved in systemic inflammation such as TNF-α (FIG. 3A) and IL-6 (FIG. 3B) significantly decreased in subjects treated by Beinaglutide when compared to the control group. A decrease in the protein level of liver collagen 1 alpha 1 (FIGS. 3C and 3E) suggested a reduction of fibrosis in the subjects' liver after Beinaglutide treatment (2.4 mg/kg). Finally, subjects treated with Beinaglutide treatment (2.4 mg/kg) showed weight loss at the beginning of the treatment compared with the negative control group, and improved weight control compared with the control group (FIG. 4A). Oral glucose tolerance test (OGTT) was the gold standard for diagnosis of type 2 diabetes. Subjects treated with Beinaglutide treatment (2.4 mg/kg) showed lower blood glucose in the OGTT compared with the control group (FIG. 4B). Lower Homeostatic Model Assessment of Insulin Resistance (HOMA-IR) results of the subjects treated with Beinaglutide treatment (2.4 mg/kg) showed lower insulin resistance than the control group (FIG. 4C). Various Beinaglutide treatments (0.6 mg/kg, 1.2 mg/kg, 2.4 mg/kg) for 11 weeks were evaluated for therapeutic effects in HFD-NASH mice (Example 2): 1) Beinaglutide significantly slowed down body weight gain (FIGS. 13A-13D) and liver weight and liver index increase (FIGS. 15A-15B), and significantly reduced appetite (FIGS. 14A-14B) of the HFD-NASH mice in all Beinaglutide treatment groups compared to the control group; 2) the serum ALT (FIG. 17A), TC levels (FIG. 17E), and liver TG content (FIG. 18B) significantly reduced in Beinaglutide 1.2 mg/kg and 2.4 mg/kg groups, and the serum LDL-c levels (FIG. 17F) and liver Co/1a1 gene expression levels (FIG. 19A) were significantly decreased in all Beinaglutide treatment groups; and 3) histopathological results showed that the liver fibrosis scores significantly decreased in Beinaglutide 0.6 mg/kg and 2.4 mg/kg treatment groups compared to that in the control group at the end of study (FIGS. 20A-20C and 21A-21C), and in Beinaglutide 2.4 mg/kg treatment group, the area of macrovesicular fat droplets in liver tissue significantly reduced (FIG. 23B) and the numbers of animals with severe grading were also decreased for steatosis, steatosis location, interlobular inflammation, liver fibrosis grades and pattern.

Thus, Beinaglutide may be a promising therapy for treating NASH. It is contemplated that the GLP-1 and/or GLP-1 analog(s) disclosed herein may also be used to treat NASH.

Disclosed herein is a method of treating or preventing NASH using a composition comprising an effective amount of one or more first active ingredients selected from the group consisting of GLP-1 and GLP-1 analogs (e.g., GLP-1 (7-36) such as Beinaglutide).

In the context of this disclosure, the phrase a “therapeutically effective amount” or an “effective amount” of a pharmaceutical composition comprising GLP-1 and/or GLP-1 analogs (e.g., GLP-1 (7-36) such as Beinaglutide) as used herein is an amount of the pharmaceutical composition that produces a desired therapeutic effect in a subject, such as treating or preventing NASH. In certain embodiments, the therapeutically effective amount is an amount of the pharmaceutical composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. In some embodiments, a therapeutically effective amount is the minimal amount that produces a therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological art will be able to determine a therapeutically effective amount through routine experimentation, namely by monitoring a subject's response to administration of the pharmaceutical composition and adjusting the dosage accordingly. For additional guidance, see, e.g., Remington: The Science and Practice of Pharmacy, 22nd Edition, Pharmaceutical Press, London, 2012, and Goodman & Gilman's The Pharmacological Basis of Therapeutics, 12th Edition, McGraw-Hill, New York, N.Y., 2011, the entire disclosures of which are incorporated by reference herein.

The terms “treat,” “treating,” and “treatment” as used herein with regard to a condition refers to alleviating the condition partially or entirely, preventing the condition, decreasing the likelihood of occurrence or recurrence of the condition, slowing the progression or development of the condition, or eliminating, reducing, or slowing the development of one or more symptoms associated with the condition.

As used herein, the term “subject” refers to a mammalian subject, preferably human. In certain embodiments, the subject has been diagnosed with NASH, or is at an elevated risk of developing NASH. The phrases “subject” and “patient” can be used interchangeably herein.

In certain embodiments, the pharmaceutical composition disclosed herein comprises a therapeutically effective amount of one or more first active ingredients selected from the group consisting of GLP-1 and GLP-1 analogs. In some embodiments, the GLP-1 analogs are selected from the group consisting of GLP-1 (7-37), GLP-1 (7-36), and GLP-1 (7-35). Unless specified otherwise, GLP-1 and a GLP-1 analog may have a C-terminal free carboxyl group or a C-terminal amide group. For example, the GLP-1 analog can be a recombinant human GLP-1 (7-36) peptide having the sequence of: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 1), which may be referred to as Beinaglutide in this disclosure. Beinaglutide has a molecular formula of C₁₄₉H₂₂₅N₃₉O₄₆, and a molecular weight of 3,298.7. Beinaglutide is essentially the same as the active form of circulating GLP-1 except for the endogenous amidation, where NH₂ in the natural form is replaced by OH group in the recombinant peptide. Beinaglutide includes a C-terminal free carboxyl group. In other embodiments, GLP-1 (7-35) or GLP-1 (7-37) can be used in the disclosed technology. The sequences of GLP-1 (7-35) having a C-terminal free carboxyl group and GLP-1 (7-37) having a C-terminal free carboxyl group are as follows:

(SEQ ID NO: 2)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly,
and
(SEQ ID NO: 3)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly.

In some embodiments, the GLP-1 and/or GLP-1 analog(s) (e.g., GLP-1 (7-36) such as Beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as Beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight.

In some embodiments, the GLP-1 and/or GLP-1 analog(s) (e.g., GLP-1 (7-36) such as Beinaglutide) may be administered once a day, twice a day, three times a day, or four times a day. In some embodiments, the total daily dosing of the GLP-1 and/or GLP-1 analog(s) (e.g., GLP-1 (7-36) such as Beinaglutide) may be about 40 μg to about 14,000 μg, about 40 μg to about 13,500 μg, about 50 μg to about 14,000 μg, about 50 μg to about 13,500 μg, about 40 μg to about 12,030 μg, about 50 μg to about 12,040 μg, about 2,010 μg to about 14,000 μg, about 1,510 μg to about 13,500 μg, about 250 μg to about 6,000 μg, about 250 μg to about 5,700 μg, about 300 μg to about 6,000 μg, about 300 μg to about 5,700 μg, about 480 μg to about 700 μg, about 480 μg to about 600 μg, about 540 μg to about 700 μg, or about 540 μg to about 600 μg.

In addition to the GLP-1 and/or GLP-1 analog(s) (e.g., GLP-1 (7-36) such as Beinaglutide), the pharmaceutical composition disclosed herein may contain one or more pharmaceutically acceptable excipients, and/or buffer (e.g., histidine-hydrochloric acid (histidine-HCl), sodium citrate-citric acid, disodium hydrogen phosphate-citric acid, NaOH-citric acid, sodium acetate-acetic acid (NaAC-HAC), succinate-succinic acid, lactate-lactic acid, glutaminate-glutamic acid, malate-malic acid, benzoate-benzoic acid, tartrate-tartaric acid or glycine-hydrochloric acid (Gly-HCl) or any combinations thereof) salt to maintain the desired pH range of the composition. Additional ingredients for the pharmaceutical composition may include one or more of preservatives (e.g., phenol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof), isotonic agents (polyol, sodium chloride, sugar or any combinations thereof; wherein, the polyol is mannitol, sorbitol, inositol, xylitol, glycerin, propylene glycol or any combinations thereof; and the sugar is sucrose, trehalose, lactose, fructose, glucose or any combinations thereof), and dissolution enhancers (e.g., Tween 20, Tween 40, Tween 80, Span 20, Span 40, Span 80, Poloxamer 188, Pluronic F68, Brij 35, dextran-20, PEG 400, PEG 1000, PEG 1500, PEG 2000, propylene glycol or any combinations thereof).

The pharmaceutical composition is formulated to be suitable for a particular administration route. For example, the pharmaceutical composition can be injected subcutaneously, intraperitoneally, or intravenously, or be administered by infusion. In some embodiments, the pharmaceutical composition can also be administered by a nasal spray or by oral administration. In some embodiments, the pharmaceutical composition comprising the GLP-1 and/or GLP-1 analog(s) (e.g., GLP-1 (7-36) such as Beinaglutide) may be delivered once a day, twice a day, three times a day or four times day. When multiple doses of the GLP-1 and/or GLP-1 analog(s) (e.g., GLP-1 (7-36) such as Beinaglutide) are administered, it is not necessary that the same dose is administered each time to the subject. It is possible to administer a first dose of the GLP-1 and/or GLP-1 analog(s) (e.g., GLP-1 (7-36) such as Beinaglutide) and then adjust the subsequent dose(s) higher or lower depending on the patient's response to the first dose.

As demonstrated in the working example, a total of 70 male ob/ob mice (6 weeks old) were introduced into SPF animal room and fed with normal rodent diet for 1 week, then fed with D0910310 high fat diet (HFD, fat (40 Kcal %), fructose (20 Kcal %) and cholesterol (2%), also named GAN (Gubra Amylin NASH) diet) for 6 weeks. Artificial inversed circadian rhythms were adopted during this induction phase and subsequent drug administration phase. The animals were randomly allocated into different groups according to body weight and plasma ALT levels at the end of week 6. Vehicle (3 mi/kg, s.c, t.i.d) and different doses (0.6, 1.2, 2.4 mg/kg, s.c, t.i.d, 3 ml/kg) of Beinaglutide were administrated from week 7 to week 17 (11 weeks in total), with the mice maintained on HFD diet in this phase. Six wild-type C57BL/6J mice as WT control were introduced into SPF animal vivarium in the first week of drug treatment. Normal artificial circadian rhythms were adopted for these WT mice during the study, with normal rodent diet and vehicle (3 ml/kg, s.c, t.i.d) dosing, and with the same examinations as that for the main study mice. Body weight and food consumption were measured twice a week. The proper study ending time point was determined by comprehensive considerations on animal status, body weight, plasma ALT level, and food consumption indexes. The end points of the study included body weight, liver weight, liver index, serum biochemical analysis (AST, ALT, TBIL, TC, TG, and LDL-c), liver TC/TG content and mRNA expression analysis for TGFa1, Col1a1 and α-SMA in liver tissues, IHC staining for α-SMA, Oil Red staining for liver lipid deposition, H&E staining for steatosis, inflammation, ballooning degeneration and fibrosis, and Sirius Red staining to quantify fibrosis stages.

The experimental results demonstrate that HFD successfully induced NASH in ob/ob mice. After about 11 weeks of continuous administration of low, medium and high doses of Beinaglutide, the body weight, liver weight, liver index, serum LDL-c level and Col1a1 gene expression level in liver tissues significantly (P<0.05, or P<0.01, or even P<0.001) decreased, and all doses decreased mice food consumption during the drug administration period. The medium dose (1.2 mg/kg) and high dose (2.4 mg/kg) of Beinaglutide administration significantly (P<0.05 or P<0.01) decreased ALT and TC levels in serum, as well as TG content in liver tissues. The low dose (0.6 mg/kg) and high dose (2.4 mg/kg) of Beinaglutide administration significantly (P<0.05) decreased liver fibrosis score. A high dose (2.4 mg/kg) of Beinaglutide administration significantly (P<0.01) decreased the area of macrovesicular fat droplets in liver tissue. For indexes of steatosis, steatosis location, interlobular inflammation, liver fibrosis scores and liver fibrosis pattern, the animal counts with severe pathological changes were decreased after 0.6 mg/kg or 2.4 mg/kg of Beinaglutide treatment. In addition, Beinaglutide at all 3 doses appeared to have decreased the serum AST level, liver inflammation score, NAS score, and liver α-SMA expression level demonstrated by IHC staining and liver fibrosis area demonstrated by Sirius Red staining.

Therefore, the Beinaglutide treatment decreased animal body weight and liver weight, reduced hepatic fat, ameliorated liver function injuries, and reduced liver inflammation and fibrosis, and demonstrated beneficial therapeutic effects on HFD induced NASH model in mice.

In certain embodiments, the treatment protocol disclosed in this document can be combined with physical exercise and/or restricted diet for controlled calorie intake.

The following examples are provided to better illustrate the claimed invention and the embodiments described herein, and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Effects of Beinaglutide on NASH Mice

This example demonstrates the pharmacokinetics and pharmacodynamics of Beinaglutide in HFD-NASH mouse models.

### Materials

C57BL/6J mice: Male C57BL/6J mice (8 weeks, old. Lot No.: C5720191021) were purchased from Shanghai Jihui Laboratory Animal Care Co., Ltd. Upon arrival, the mice were housed in animal facility in Shanghai University of Medicine & Health Sciences, with constant temperature (20-24° C.) and humidity (40-70%). Housing was provided a 12:12-hour light-dark cycle. The mice were allowed ad libitum access to chow diet.

HFD-NASH model development Animals were group-housed in standard cages at 22° C. in a 12:12-h light-dark cycle. Ob/ob mice were allowed ad libitum access to a diet enriched in fat (40% kcal), fructose (20% by weight), and cholesterol (2% by weight) (Research Diets, Inc., #D09100310). Over 9 weeks feeding, the mice (HFD-NASH) were developed with nonalcoholic fatty liver disease (steatosis, steatohepatitis with fibrosis), as assessed by biochemical methods (e.g., ALT levels).¹ ¹Santhekadur P K, Kumar D P, Sanyal AszJ. Preclinical models of non-alcoholic fatty liver disease. J Hepatol. 2018 February; 68(2):230-237; 2) Clapper J R. Hendricks M D, Gu G. at al. Diet-Induced mouse model of fatty liver disease and nonalcoholic steatohepatitis reflecting clinical disease progression and methods of assessment. Am J Physiol Gastrointest Liver Physiol 305: G483-G495. 2013.

Active agents: Beinaglutide used in this example had the sequence of His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 1) and was obtained from Benemae (Beinaglutide injection, 2 mg/mL), and stored at 2-8° C.

Vehicle: The vehicle used in this example was the same as the Beinaglutide injection from Benemae except that the vehicle does not include Beinaglutide. The vehicle was an aqueous solution that included mannitol, propylene glycol, phenol (2.00-2.40 mg/ml), acetic acid, sodium acetate, and water for injection.

### Pharmacokinetics Study of Beinaglutide

Mice (n=4 per time point) were randomized into different groups by body weight on the day before the study. Mice were fasted overnight before dosing. Before Beinaglutide subcutaneous dosing, baseline blood was collected from orbital sinus. Samples were collected at the time intervals shown in Table 1, where about 60 μL blood was taken into EDTA-coated tubes (pre-added 2.5 μL DPP-4 inhibitor, and 2.5 μL protease inhibitor). The plasma was separated on a pre-cooled centrifuge and then immediately frozen in dry ice. The samples were stored at −80° C. till assay. Plasma GLP-1 (7-36) was analyzed with ELISA kit (EMD Millipore, Cat. #EZGLPHS-35K). After 9 weeks diet-induction, the mice remained on high-fat/high-cholesterol/high-fructose diet (D09100310).

TABLE 1
Blood collection time points for HFD-NASH mice

| Time (min) | Animal No. |
|---|---|
| 0 | 1-4 |
| 5 | 5-8 |
| 10 | 9-12 |
| 20 | 13-16 |
| 30 | 1-4 |
| 45 | 5-8 |
| 60 | 9-12 |
| 90 | 13-16 |
| 120 | 1-4 |
| 180 | 5-8 |
| 240 | 9-12 |

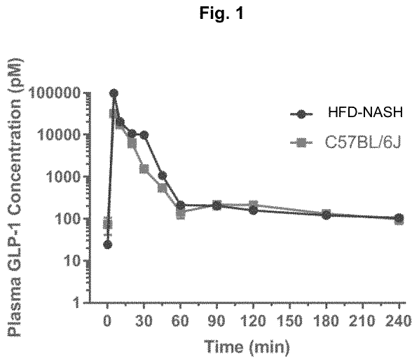

The pharmacokinetic data is shown in FIG. 1. No significant difference of pharmacokinetics was observed between the C57BL/6J mice and HFD-NASH mice.

### Pharmacodynamics Study of Beinaglutide

Studies on HFD-NASH mice: The study design for HFD-NASH mice is shown in Table 2 below.

TABLE 2
Study Design for HFD-NASH Mice

| | | | | Dose volume | |
|---|---|---|---|---|---|
| No. | Groups | n | Treatment | (mL/kg) | Frequency |
| 1 | Control | 8 | Vehicle | 3 | 3 times a day |
| 2 | Treatment | 8 | Beinaglutide | 3 | 3 times a day |
| | | | at 2.4 mg/kg | | |

After 9 weeks diet-induction, HFD-NASH mice were randomized into different groups by body weight. Body weight and diet were measured every day. At termination, blood was collected by cardiac puncture after animal's euthanasia by CO₂. Liver were harvested and weighted. Right medial and/or left lateral lobes of the liver were excised. One part of liver was frozen for liver triglyceride measurement. One part was frozen for protein, RNA level qualification, like collagen 1 alpha 1 (Col1a1). One part was paraffin embedded. Hematoxylin and eosin (H&E) were used for morphological analysis. Picrosirius red stains was used for assessment of hepatic fibrosis. Histopathological analysis was performed by a pathologist blinded to the study. The remaining liver tissue was kept at −80° C. till analysis. Plasma levels of ALT, AST, TG, and total cholesterol (TC) were measured by bioanalyzer or kits. After 9 weeks diet-induction, the mice remained on high-fat/high-cholesterol/high-fructose diet (D09100310).

Liver Steatosis

Beinaglutide treatment significantly reduced liver steatosis in HFD-NASH mice (FIGS. 2A-2E). The animals of two groups were subcutaneously injected (3 mL/kg) three time per day for 4 weeks with vehicle (Vehicle) or GLP-1 (7-36) (2.4 mg/kg). Liver weight (FIG. 2A), liver triglyceride (TG) content (FIG. 2B), plasma alanine aminotransferase (ALT, FIG. 2C) and plasma aspartate aminotransferase (AST, FIG. 2D) were measured at termination, which were significantly reduced in the treatment group compared to the control group. Representative hematoxylin and eosin (H&E)-stained liver sections showed lipid accumulation in the vehicle and Beinaglutide (2.4 mg/kg) group (FIG. 2E). In the Beinaglutide group, macrovesicular (stars) and microvesicular (arrows) steatosis were significantly reduced. *P<0.05, **P<0.01 vs vehicle group by student t test.

Liver Inflammation

Beinaglutide treatment significantly reduced plasma levels of inflammation markers in HFD-NASH mice (FIGS. 3A-3B). The animals of two groups were subcutaneously injected (3 mL/kg) three time per day for 4 weeks with vehicle (Vehicle) or Beinaglutide (2.4 mg/kg). Plasma TNF-α (FIG. 3A) and IL-6 (FIG. 3B) levels were measured by commercial kits, which were involved in systemic inflammation and significantly reduced in the treatment group.

Liver Fibrosis

Beinaglutide treatment (2.4 mg/kg) significantly reduced a fibrosis marker (liver collagen 1 alpha 1) in HFD-NASH mice (FIGS. 3C-3D) and reduced hepatic fibrosis (FIG. 3E). Protein level of liver collagen 1 alpha 1 was determined by western blotting (FIG. 3C). The corresponding densitometry analysis was calculated and normalized to R-actin, which was significantly lower in the treatment group (FIG. 3D). Liver sections were stained with picrosirius red for collagen (FIG. 3E). Hepatic fibrosis was indicated by black triangles (A) and significantly reduced in the treatment group. ***P<0.001 vs vehicle group by student t test.

Weight Control and Insulin Resistant

Beinaglutide treatment (2.4 mg/kg) also improved weight control (FIG. 4A) and reduced insulin resistant (FIGS. 4B and 4C) in subjects compared with the control group. The animals of two groups were subcutaneously injected (3 mL/kg) three time per day for 4 weeks with vehicle (Vehicle) or Beinaglutide (2.4 mg/kg). Body weight change was recorded every day. The subjects treated with Beinaglutide had a lower body weight compared with the control group and showed an improved weight control (FIG. 4A).

Oral glucose tolerance test (OGTT) was performed at day 30 and was a test to diagnose type 2 diabetes (FIG. 4B).

Homeostatic Model Assessment of Insulin Resistance (HOMA-IR) is a calculation marker for insulin resistance which was calculated by formula: Glucose (mM)×Insulin (μM/L)/22.5 (FIG. 4C). *P<0.05, *P<0.01 vs vehicle group by student t test. Subjects treated Beinaglutide (2.4 mg/kg) showed lower insulin resistance than the control group (FIG. 4C). Thus, Beinaglutide treatment improved weight control of the subjects treated and lowered the risks for type 2 diabetes and insulin resistance.

Thus, Beinaglutide treatment appeared to be a promising therapy for treating NASH.

### Example 2: Effects of Beinaglutide on Non-Alcoholic Steatohepatitis HFD (NASH) Mice

This example demonstrates the therapeutic effects of Beinaglutide on high fat diet (HFD) induced NASH model in mice.

### Materials and Methods

Animals: Seventy ob/ob mice (B6.V-Lep^{ob}/J) were purchased from Beijing HFK Bioscience Co., Ltd. (22 backups). Six C57BL/6J mice were purchased from Jiangsu GemPharmatech, Co., Ltd. Male, 6-week old mice having a naïve dosing record were used. The mice had free access to water and free access to rodent diet treated by irradiation sterilization. The animals were received and transferred to the cages upon arrival. Then their appearance, limbs and orifices of each mouse were examined, and their posture or behavior abnormalities were checked. The animals in good conditions were acclimated for 1 week prior to being put on the study.

The mice were housed in cages (260 mm×160 mm×120 mm) in animal room, 3-5 animals per cage. The padding was corn cob crumbs that had been sterilized at high temperature (provided by HDB), changed once a week. The frequency of filtered ventilation in the animal room was 15-25 times per hour. The temperature was kept at 20-26° C. (68-79° F.), and relative humidity at 40-70%. The animals were acclimated for one week under normal artificial circadian rhythms, then were switched to inversed artificial circadian rhythms produced by fluorescent lighting, except for the C57BL/6J mice, from the 1st day of model induction to the end of in vivo experiment (12-hour light/12-hour dark cycle, except for the time of dosing).

Experimental mice were allowed free access to different diets (sterilized by irradiation). During the entire experimental period, the mice were allowed free access to drinking water.

Active agents: An injectable formulation of Beinaglutide (4.2 mg/2.1 mL, 42000U) was obtained from Benemae (stored at 2-8° C. until use). Beinaglutide was injected subcutaneously with the original injection solution, or with a solution of a suitable concentration diluted with the vehicle, which only differed from the Beinaglutide injectable formulation by not having Beinaglutide. The vehicle was an aqueous solution that included mannitol, propylene glycol, phenol (2.00-2.40 mg/ml), acetic acid, sodium acetate, and water for injection.

Animal grouping and experiment process: A total of 70 male ob/ob mice (6 weeks old) were fed with a chow diet after transferred to SPF animal room and acclimatized for 1 week. The feed was then switched to HFD diet (D09100310, fat (40 Kcal %), fructose (20 Kcal %) and cholesterol (2%), also named GAN (Gubra Amylin NASH) diet, Research Diets, Inc., USA) and maintained for 6 weeks. Inversed artificial circadian rhythms lighting were adopted during this period and subsequent drug administration period.

Plasma samples were collected for the measurement of ALT and mouse body weight data were measured at week 6. Animals were then randomly allocated into 4 groups (12 animals/group) based on body weight (the first weight index) and plasma ALT level (the second weight index). Drug and vehicle administration were started from week 7 since modeling start and mice were maintained on HFD diet with inversed artificial circadian rhythms lighting.

A total of 6 male C57BL/6J mice (7-week old, wild type) were introduced into SPF animal room (the artificial circadian rhythms are normal) as WT control group within the 1^{st} week of drug administration. C57BL/6J mice were fed with normal rodent chow diet and dosed with equivalent volume of vehicle since other mice were dosed at week 3 (see Table 3 below).

TABLE 3
Animal Grouping

| Group | | | |
|---|---|---|---|
| No. | Description | N | Treatment |
| 1 | WT control (C57BL/6J) | 6 | Vehicle 3 ml/kg, t.i.d, |
| | | | s.c., 9 weeks |
| 2 | HFD-NASH control | 12 | Vehicle 3 ml/kg, t.i.d, |
| | | | s.c., 11 weeks |
| 3 | Beinaglutide low dose | 12 | Beinaglutide 0.6 mg/kg, |
| | | | t.i.d, s.c., 11 weeks |
| 4 | Beinaglutide medium dose | 12 | Beinaglutide 1.2 mg/kg, |
| | | | t.i.d, s.c., 11 weeks |
| 5 | Beinaglutide high dose | 12 | Beinaglutide 2.4 mg/kg, |
| | | | t.i.d, s.c., 11 weeks |

For ALT monitoring, plasma collection methods were as follows: The mice were briefly anesthetized with isoflurane. About 150 μl of whole blood of each mouse was collected from the orbital venous into one tube with anti-coagulant EDTA-K. Plasma were obtained by centrifugation at 3000 rpm at 4° C.

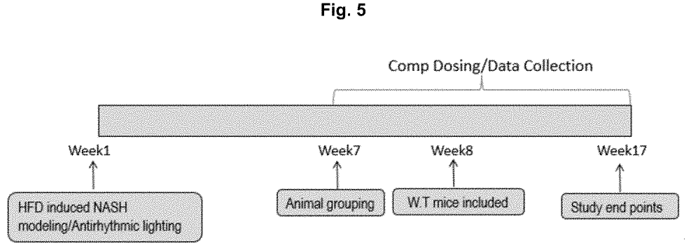

The experiment flow chart is shown in FIG. 5. The general conditions of the mice were examined daily by a veterinarian.

Data recording: Appearance and behavior of each mouse were observed after administration. All abnormal appearance, status and behaviors were recorded. In the period of HFD model development, body weight was measured once weekly from week 1 to week 6. Then body weight and food consumption were measured twice weekly since compound dosing start (week 7). The food consumption was calculated by the diet consumption of each cage of mice within 8 hours per day, the average food consumption per group was calculated. The plasma samples were collected at 4, 7 and 10 weeks after compound dosing and used for the analysis of ALT to determine the appropriate time for the end of drug administration.

Blood and tissue sampling: At the end of the in vivo study, all mice were euthanized via carbon dioxide (CO₂) asphyxia after 11.5 hours fasting. Blood samples were collected via cardiac puncturing, one aliquot of each blood sample was collected into a tube without anti-coagulant for serum preparation. Serum was obtained by centrifugation at 3000 rpm for 10 minutes at 4° C. and then stored in a −80° C. refrigerator before being used for the measurement of ALT, AST, total bilirubin (TBIL), TC, TG and LDL-c. The other aliquot of each blood sample was collected into a tube with EDTA-K anti-coagulant for plasma preparation. Plasma samples were obtained by centrifugation at 4000×g for 10 minutes at 4° C. and then stored in a −80° C. refrigerator.

After the animal was sacrificed, the body weight and liver weight were measured, and four lobes of each liver were collected. The right middle lobe of each liver was collected, cut into two sections, put into two SNAP frozen tubes and kept at −80° C. before testing. One section of the liver tissue was used for mRNA expression analysis of Tgfb1, Col1a1 and Acta2; and the other section of the liver tissue was used for the measurement of TC and TG contents. The left middle lobe of each liver was collected, store at −80° C. for future use. The left lateral lobe of each liver was collected, cut into two sections and put into two tubes. One section was used for optimal cutting temperature (OCT) embedding, frozen sectioning and oil red staining for steatosis examination, and for calculation of percentage of fat deposition area. The other section was fixed with 10% neutral formalin solution and processed into formalin fixed paraffin-embedded (FFPE) block for H&E, Sirius red or α-SMA IHC staining respectively. The right lateral lobe of each liver was collected and fixed in 10% neutral formalin for future use.

Serum biochemistry analysis: The serum samples were taken out from −80° C. refrigerator and restored to room temperature. 150 μl of each serum sample was pipetted out for the analysis of ALT, AST, TBIL, TC, TG and LDL-c with automatic biochemical analyzer (HITACHI 7180).

qPCR analysis: The right middle lobe of each liver tissue was cut into small pieces, and 30-50 mg of liver tissue was weighed and transferred into a Lysing Matrix D tube, then 1 ml of TRIzol Reagent was added into Lysing Matrix D containing tissue immediately. The Lysing Matrix D tubes containing tissue and TRIzol reagent was placed into the Fast Prep-24 Sample Preparation System (Shanghai Jingxin Science and Technology Co., Ltd.), ground for 2 minutes at a speed setting of 6. The homogenate was transferred into a new tube. The RNA samples were prepared, the reverse transcription was carried out, and the real time PCR was performed in 384-well plates according to standard protocols. The primer sequences are shown in Table 4 below.

TABLE 4
Primer Sequences for QT-PCR

| Target | Product | |
|---|---|---|
| Gene | Length (bp) | Primer Sequence (5′ → 3′) |
| Gapdh | 104 | Forward-CATGGCCTTCCGTGTTCCTA |
| | | (SEQ ID NO: 4) |
| | | Reverse-CCTGCTTCACCACCTTCTTGAT |
| | | (SEQ ID NO: 5) |
| Col1a1 | 141 | Forward-CTGGCGGTTCAGGTCCAAT |
| | | (SEQ ID NO: 6) |
| | | Reverse-TTCCAGGCAATCCACGAGC |
| | | (SEQ ID NO: 7) |
| Acta2 | 104 | Forward-CCCAGACATCAGGGAGTAATGG |
| | | (SEQ ID NO: 8) |
| | | Reverse-TCTATCGGATACTTCAGCGTCA |
| | | (SEQ ID NO: 9) |
| Tgfb1 | 142 | Forward-CTTCAATACGTCAGACATTCGGG |
| | | (SEQ ID NO: 10) |
| | | Reverse-GTAACGCCAGGAATTGTTGCTA |
| | | (SEQ ID NO: 11) |

The data was expressed as mean±SEM. The 2-ΔΔCT method was used to analyze the mRNA expression level. The results were normalized for RNA input using glyceraldehydes-3-phosphate dehydrogenase (GAPDH). Statistical analysis was performed using one-way ANOVA followed, if significant, by post-hoc Dunnett's test. Non parametric tests like Mann-Whitney were used when the N was too small or data did not follow Gaussian location. The difference was considered significant when P<0.05.

Liver lipid content analysis: The second piece of the right middle lobe of the liver was removed from −80° C. refrigerator, with 8 μL of isopropanol per 1 mg tissue added, and heated for 5 minutes at 80° C., cooled down at room temperature. Then 12 μL hexane per 1 mg tissue was added, homogenized at 3000 rpm for 7 minutes at room temperature, and supernatant was pipetted out. Equal volume of 67 mg/mL Na₂SO₄ solution to the supernatant was added, mixed for 1 minute, allowed to separate into layers, and the supernatant was pipetted out, dried overnight in fume hood, and tertiary butanol:methanol (3:2) solution was added to dissolve the pellets to obtain the lipid extraction samples. TC and TG contents were evaluated by a commercial kit according to the manufacturer's instructions.

Sirius red staining: FFPE blocks were cut at 4 μm in thickness, dried in an oven for 1 hour and stained with Picro Sirius Red according to HDB standard protocol. Briefly, the sections were stained with Picro Sirius Red (Head, Cat #26357, Beijing) for 90 minutes at room temperature after dewaxing and rehydration, and then dehydrated and coverslip mounted for subsequent image analysis. For image analysis of collagen deposition, Picro Sirius Red stained slides were scanned by using Aperio Scan Scope Model CS2 (Leica), at 200× magnification. Images were then opened with HALO. Using the pen tool, the whole liver section was selected as an annotation layer. The vessels were excluded in the annotation layer. The area occupied by collagen fibers was measured using HALO v2.3 software. The percentage of fibrosis (positive areas) in the selected annotation was then calculated by the “Area Quantification” module. The fibrosis was expressed as percentage per liver section.

Oil red staining: Liver frozen sections were embedded in OCT compound, cut at 7 μm in thickness and dried in room temperature for 10 minutes. The sections were flushed with deionized water and then dehydrated with propylene glycol, stained with oil red dye solution (Sigma, Lot #01516) for 5 minutes in 60° C. oven. Then the slides were transferred into 85% propylene glycol for 5 minutes and flushed with double-distilled water. Lastly the slides were stained with hematoxylin for 30 seconds followed by tap water flushing, coverslip mounting. For image analysis of fat deposition, the stained slides were scanned by using Aperio Scan Scope Model CS2 (Leica), at 200× magnification. Image files were then opened and analyzed with HALO software.

IHC staining: For IHC staining, 4 μm thick-sections from FFPE blocks were placed on slides and after overnight drying, the paraffin was removed by xylene. Then sections were placed in a graded ethanol series and finally immersed in distilled water for rehydration. After heat-induced antigen retrieval in sodium citrate solution (pH 6.0), sections were incubated in 3% hydrogen peroxide solution for 5 minutes. To avoid nonspecific staining, the sections were then incubated in blocking serum (DAKO #X0909) for 15 minutes at room temperature. Primary rabbit polyclonal anti-α-SMA antibodies (Abcam #ab5694) in 1:400 dilution were then added and incubated at room temperature for 1 hour. Finally, secondary goat polyclonal antibodies conjugated to HRP (DAKO #K4003) were added and incubated at room temperature for 30 minutes, followed by DBA color development. Counterstaining, dehydration, hyalinization and mounting were processed to get a completed slide. For image analysis of fibrosis, α-SMA stained sections were used and scanned with Aperio CS2 Scan machine. Images were then opened with HALO software. Using the pen tool, the whole liver section was selected as an annotation layer. The vessels were excluded in the annotation layer. The area occupied by collagen fibers was measured using “Area Quantification v2.1.3” module. The percentage of fibrosis (positive areas) in the selected annotation was then calculated by the program. The fibrosis was expressed as percentage per liver section.

Histopathology evaluation criteria: All histopathological scores were obtained in the whole visual field with double-blind method to ensure the accuracy and reliability of evaluation data. The specific scoring criteria are listed in Tables 5 and 6 below. After all the scores were acquired, unblinding was carried out and the data of each group were analyzed.

TABLE 5
Pathology Evaluation Criteria

| | | Ballooning | | | | Liver | |
|---|---|---|---|---|---|---|---|
| Inflammation | | Degeneration | | Steatosis | | Fibrosis Severity | |
| None | 0 | None | 0 | <5% | 0 | None | 0 |
| <2 foci | 1 | Few | 1 | 5-33% | 1 | Perisinusoidal | 1 |
| per lobule | | | | | | or Periportal | |
| 2-4 foci | 2 | Many | 2 | 34-66% | 2 | Perisinusoidal and | 2 |
| per lobule | | | | | | Portal/Periportal | |
| >4 foci | 3 | n/a | n/a | >66% | 3 | Bridging Fibrosis | 3 |
| per lobule | | | | | | | |

Nash Activity Score (NAS) = total scores of inflammation + ballooning degeneration + steatosis

TABLE 6
Indication Evaluation

| | Steatosis | Fibrosis | Microvesicuiar | Macrovesicular | Biliary |
|---|---|---|---|---|---|
| | Location | Pattern | Steatosis | Steatosis | Hyperplasia |
| None (0) | — | — | <5% | <5% | — |
| Mild (1) | Perisinusoidal | Perisinusoidal | 5-33% | 5-33% | Few biliary |
| | or Periportal | or Periportal | | | hyperplasia |
| Moderate | Portal and | Focal fibrosis | 34-66% | 34-66% | Many biliary |
| (2) | Perisinusoidal | | | | hyperplasia |
| Severe (3) | Azonal | Bridging | >66% | >66% | Most biliary |
| | | fibrosis | | | hyperplasia |

Statistical analysis: Results were expressed as mean±SEM. Statistical analysis was performed using one-way ANOVA or two-way ANOVA, and if significant, followed by post-hoc Dunnett's test. Non parametric tests like Mann-Whitney were used when the N was too small or data did not follow Gaussian distribution. The difference was considered significant when P<0.05, and very significant when P<0.01. Outlier data, if any, was excluded using Prism software (www.graphpad.com/quickcalcs/Grubbsl.cfm).

### Results

NASH Model Development

At the end of the study, the mice were euthanized after fasting for about 11.5 hours and the data or tissue samples were collected. Table 7 lists all the statistical data of HFD-NASH mouse model after 17 weeks of HFD feeding.

TABLE 7
HFD-NASH Mice Index

| Test index | | C57BL/6J | HFD-NASH |
|---|---|---|---|
| (mean ± SEM) | | Mice | Mice |
| Body Weight (g) | | 24.20 ± 0.45 | 54.97 ± 0.75 *** |
| Liver Weight (g) | | 0.88 ± 0.02 | 5.10 ± 0.13 *** |
| Liver Index (%) | | 3.61 ± 0.03 | 9.28 ± 0.19 *** |
| Serum | ALT (U/L) | 30 ± 2 | 1056 ± 63 *** |
| biochemical | AST (U/L) | 81 ± 8 | 701 ± 51 *** |
| analysis | TBIL (μmol/L) | 0.80 ± 0.13 | 2.29 ± 0.20 *** |
| | TC (mmol/L) | 2.52 ± 0.09 | 10.89 ± 0.30 *** |
| | TG (mmol/L) | 0.62 ± 0.12 | 0.42 ± 0.05 |
| | LDL-c (mmol/L) | 0.14 ± 0.03 | 1.45 ± 0.09 *** |
| Hepatic | TC (mg/g) | 1.57 ± 0.16 | 11.62 ± 0.22 *** |
| lipid analysis | TG (mg/g) | 20.95 ± 4.28 | 63.48 ± 1.86 *** |
| mRNA relative | Col1a1 | 1.00 ± 0.08 | 8.61 ± 0.72 *** |
| expression | Tgfb1 | 1.00 ± 0.02 | 1.45 ± 0.06 *** |
| level in liver | Acta2 | 1.00 ± 0.13 | 1.07 ± 0.18 *** |
| H&E staining | Inflammation | 0.0 ± 0.0 | 1.8 ± 0.2 *** |
| scores | Ballooning | 0.0 ± 0.0 | 0.5 ± 0.2 *** |
| | Degeneration | | |
| | Steatosis | 0.0 ± 0.0 | 3.0 ± 0.0 *** |
| | NAS score | 0.0 ± 0.0 | 5.3 ± 0.3 *** |
| | Fibrosis score | 0.0 ± 0.0 | 2.7 ± 0.1 *** |
| S&R staining | % S&R positive | 0.08 ± 0.02 | 2.03 ± 0.25 *** |
| | area/total area | | |
| IHC | % α-SMA | 0.09 ± 0.01 | 0.32 ± 0.04 *** |
| | positive | | |
| | area/total area | | |
| Oil red | % Oil red | 1.05 ± 0.49 | 29.17 ± 2.71 *** |
| staining | positive | | |
| | area/total area | | |

*** P < 0.001 vs C57BL/6J mice

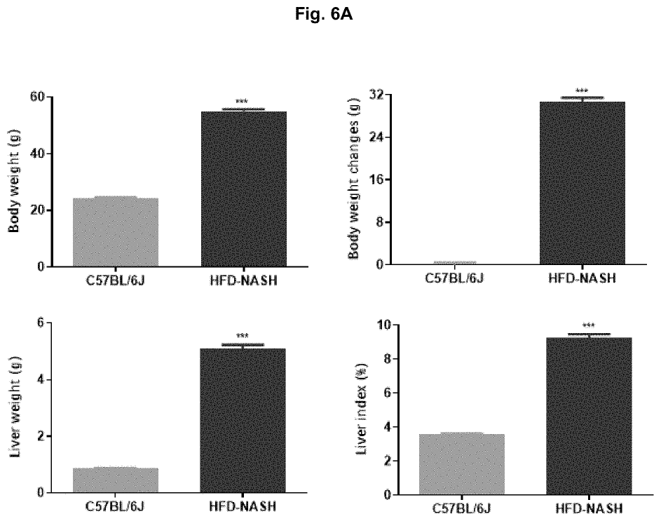

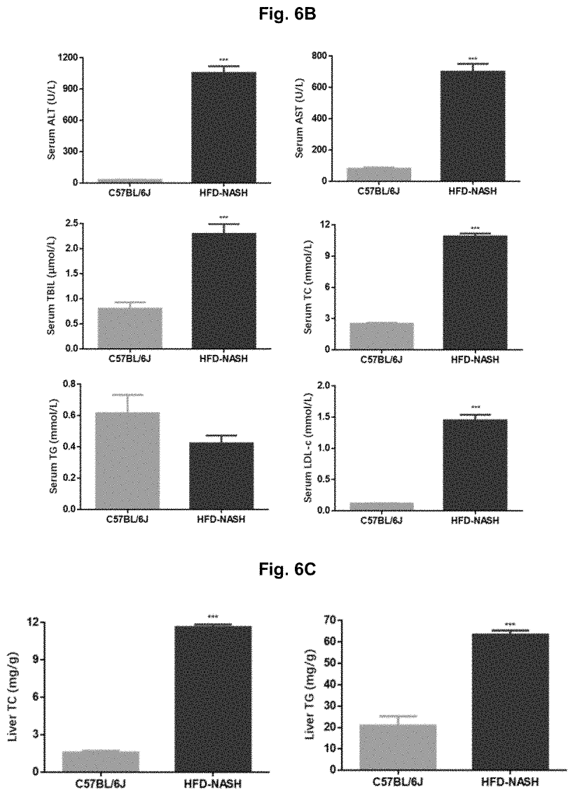

At the end of the study, when compared with C57BL/6J group, body weight, body weight change vs. baseline, liver weight and liver index were significantly increased in HFD-NASH group (FIG. 6A); ALT, AST, TBIL, TC and LDL-c level in serum were significantly elevated (with ALT and AST levels were about 35 and 9 folds of that in C57BL/6J mice, respectively), while serum TG level was consistent with the reported data (FIG. 6B); and liver TC and TG content were both significantly increased (FIG. 6C).

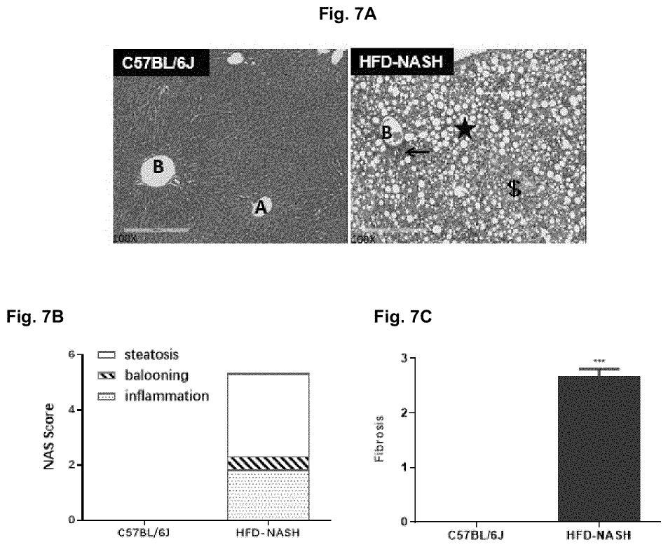

H&E staining of liver tissue showed that inflammatory cells infiltration was increased in hepatic lobules and portal areas, liver steatosis and ballooning degeneration were significantly enhanced, macrovesicular steatosis and NAS scores were significantly elevated in liver tissues from HFD-NASH group, when compared with C57BL/6J group (FIGS. 7A-7B).

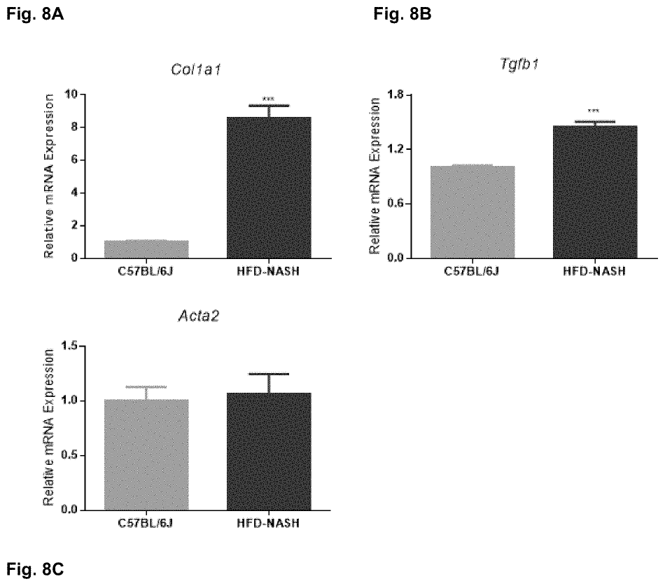

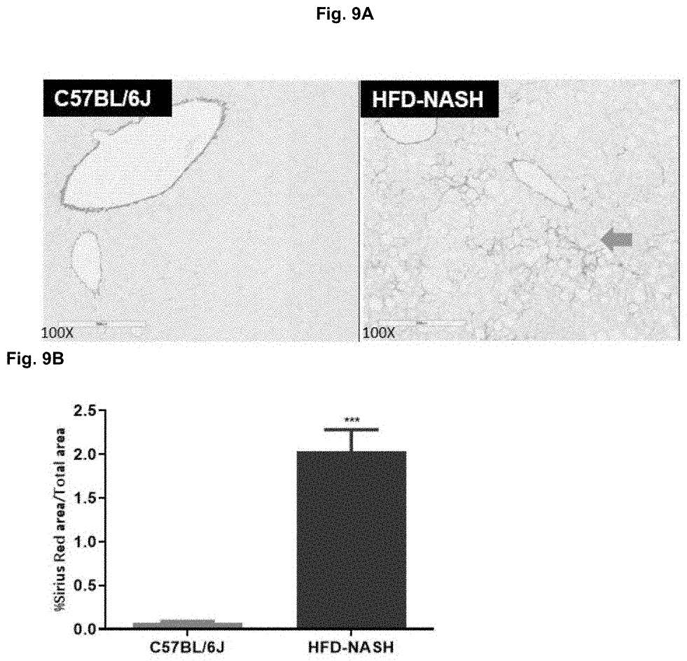

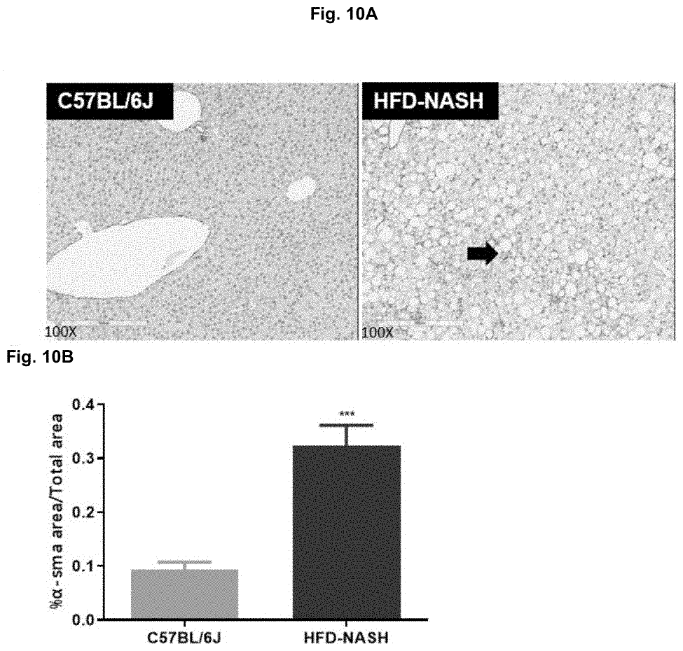

The mRNA expression levels of fibrosis related genes, Tgfb1 and Col1a1, were significantly increased in HFD-NASH mice livers (FIGS. 8A-8B). The percentage of Sirius red staining positive area and α-SMA IHC staining positive area were significantly increased in HFD-NASH group when compared with C57BL/6J group (FIGS. 9A-9B and 10A-10B). These features indicated that the liver fibrosis deposition increased, the degree of stellate cells activation significantly increased, and therefore, the severity of liver fibrosis also increased in HFD-NASH control, which was verified by the increase of fibrosis scores (FIG. 7C).

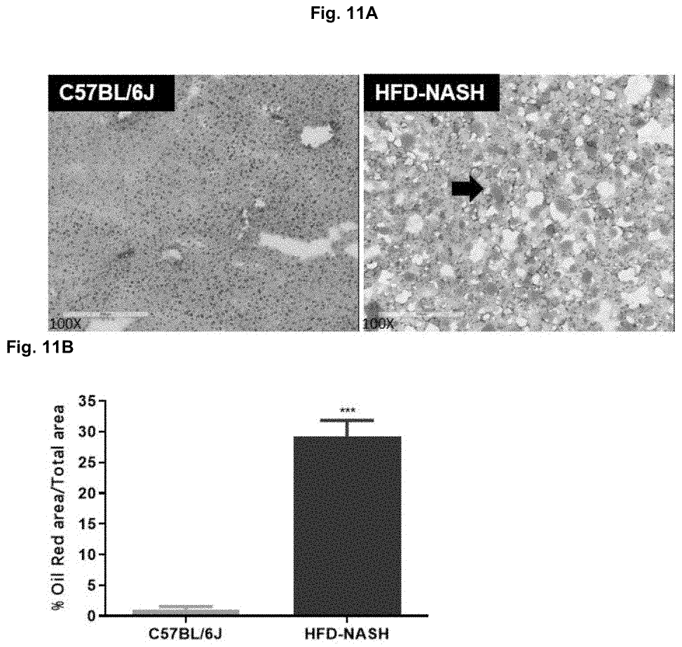

Oil red staining of liver tissue indicated that the percentage of oil red staining positive area was increased in the HFD-NASH group compared with that in the C57BL/6J group, suggesting that the liver lipid deposition and hepatic steatosis severity were significantly increased (FIGS. 11A-11B).

In sum, after 17-week HFD diet induction, obvious symptoms of NASH were observed in HFD-NASH mice, including obesity, elevated aminotransferase levels, liver steatosis, Interlobular inflammation, ballooning degeneration and fibrosis of liver tissue compared to WT C57BL/6J mice fed with normal rodent diet. Therefore, HFD diet successfully induced NASH mouse model. In this study, which can be used for pharmacodynamic evaluation of drugs for NASH treatment.

Effects on Body Weight

The WT C57BL/6J control group was not included in the following descriptions. In Beinaglutide 0.6 mg/kg group, one mouse died of bite injury during the experiment, and therefore the data of this mouse was not included in the report after its death.

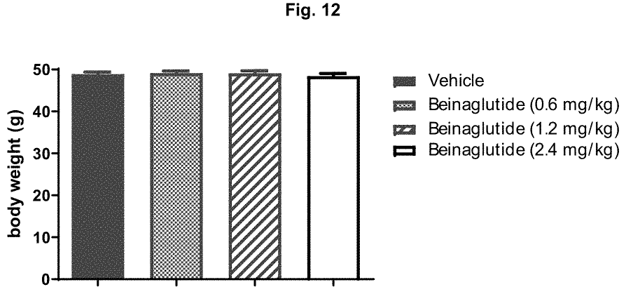

Dosing started after HFD induction for 6 weeks, and there was no significant difference in body weight among groups before dosing (FIG. 12). After treatment started, there was a much slower increase in body weight of Beinaglutide treatment groups compared with that of the control group (non-fasting body weight during drug treatment period). The mice were euthanized after fasting for 11.5 hours at the end of the study (i.e., Day 78, after 11-week drug treatment), with body weight and liver weight recorded, tissue and blood samples collected. Beinaglutide treatment significantly reduced body weight increase in comparison with that of the control group, with the percentage of body weight changes from baselines being −10.32%, −10.12% and −10.74%, respectively. These results suggest that Beinaglutide treatment inhibited body weight gain of HFD-NASH mice (FIGS. 13A-13D).

Effects on Food Consumption

Food consumption of 8 hours for each cage of the mice was recorded twice weekly after Beinaglutide treatment started, and the average food consumption for each group was calculated. The 8-hour average food consumption data of each group in the last 3 measurements towards the study end (Day 68, Day71 and Day 75) were recorded and calculated. These results indicate that the food consumption in Beinaglutide treatment groups was significantly reduced compared with that of the control group (FIG. 14), indicating that Beinaglutide administration can reduce mice food consumption and has a suppressive effect on appetite.

Effects on Liver Weight and Liver Index

At the end of the in vivo study, animals were euthanized by CO₂ asphyxiation. The liver weight and liver index significantly decreased in every Beinaglutide treatment group when compared with the control group (FIGS. 15A-15B).

Effects on Liver Function and Lipid Metabolism

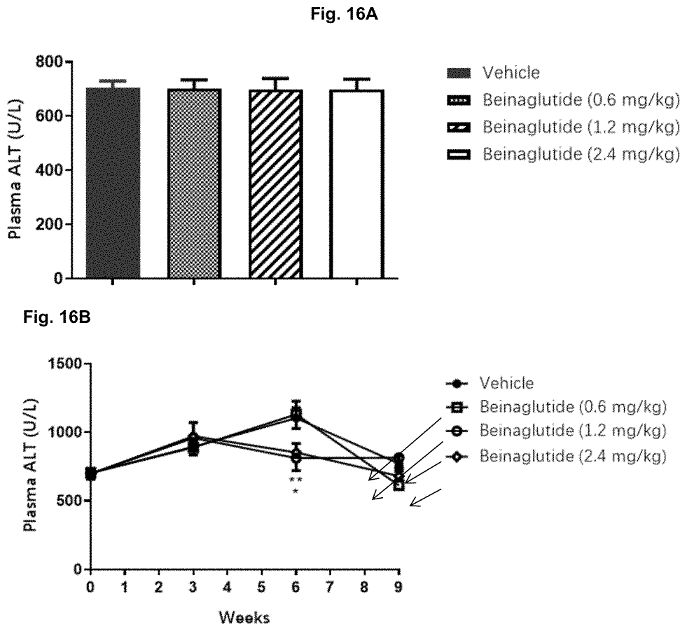

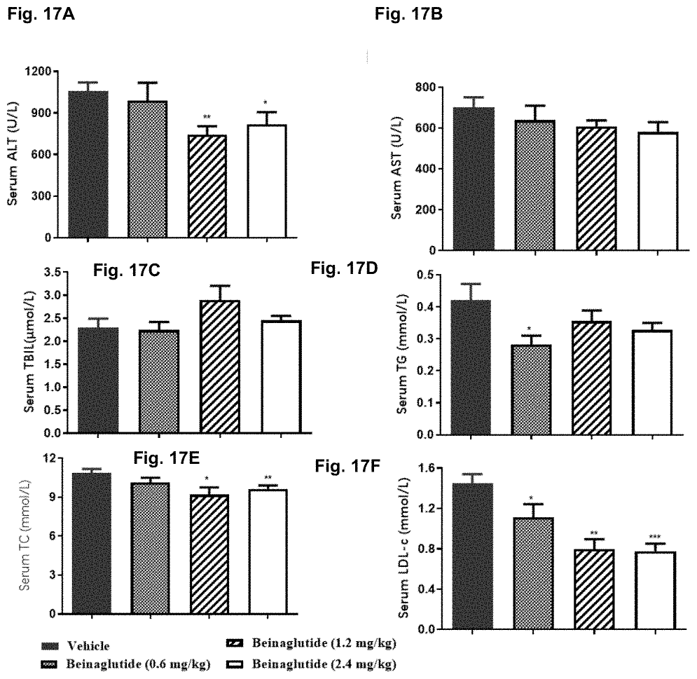

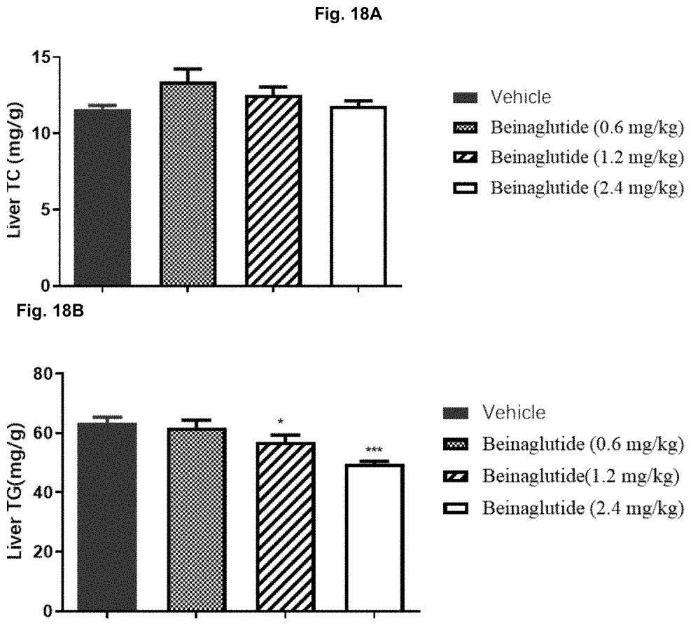

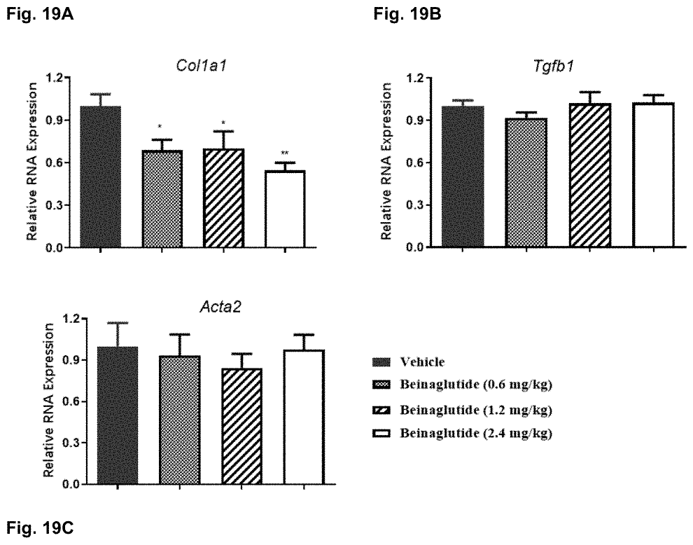

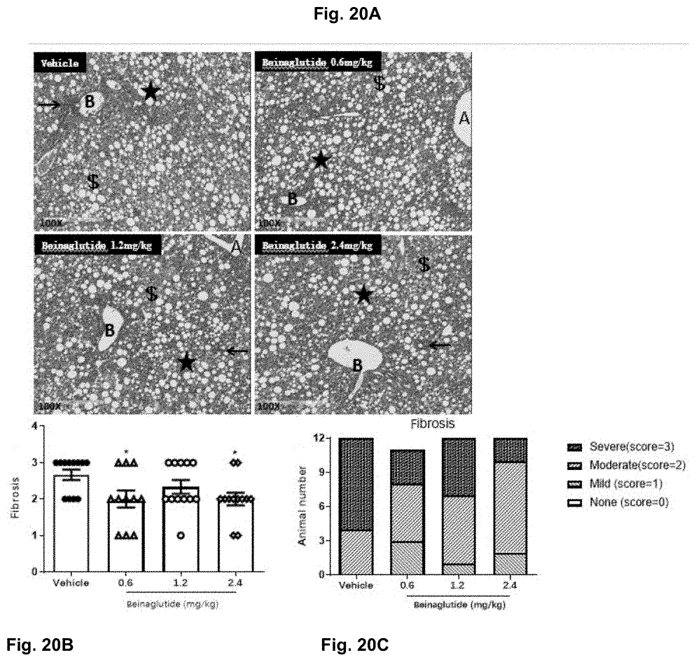

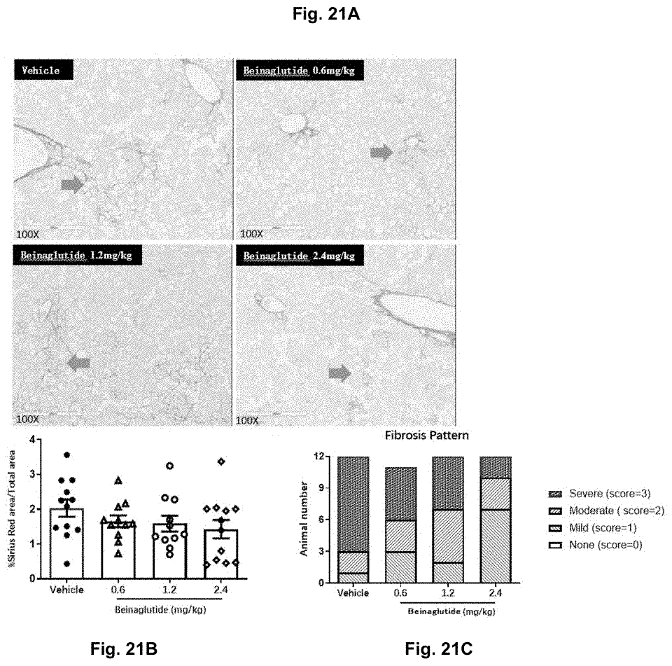

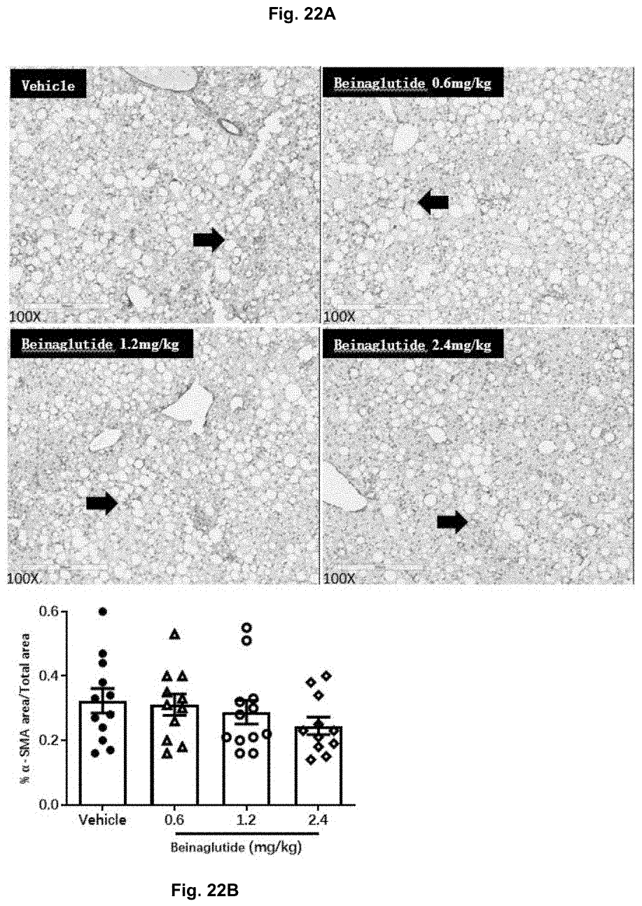

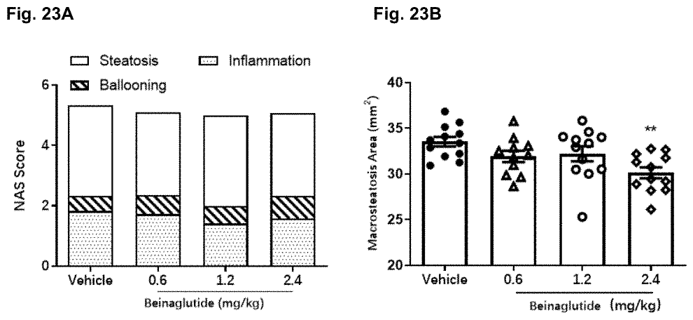

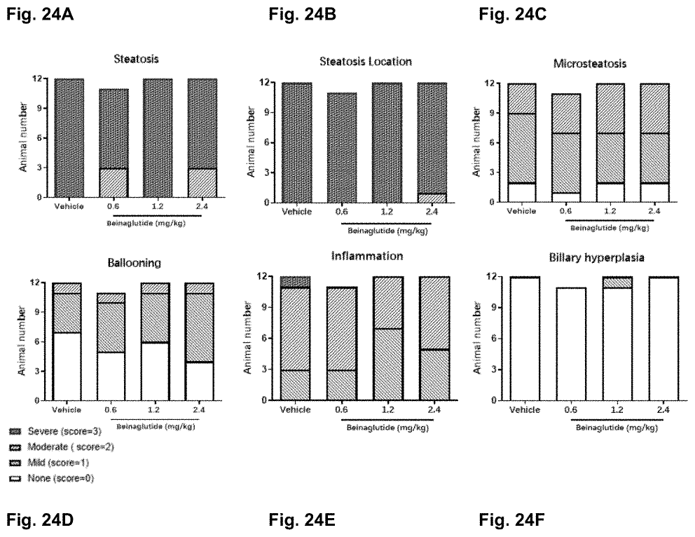

The mice were randomly assigned to different groups based on body weight and plasma ALT level after HFD induction for 6 weeks. The plasma ALT levels had no significant difference among different groups before Beinaglutide treatment (FIG. 16A). After 6 weeks of treatment, Beinaglutide 1.2 mg/kg and 2.4 mg/kg treatments significantly decreased plasma ALT level, while no significant difference between the control group and Beinaglutide treatment groups was observed after 9 weeks treatment (FIG. 16B). The lack of difference may have been due to improper detection operations because all corresponding ALT data seemed to be lower than that of week 6. The test sample was changed from plasma to serum in the subsequent experiments.

Beinaglutide 1.2 mg/kg and 2.4 mg/kg groups significantly (P<0.05, or P<0.01) decreased serum ALT (FIG. 17A), TC levels (FIG. 17E) after 11 weeks of treatment (week 17) when compared with the control group, and appeared to have decreased serum AST level (P>0.05) (FIG. 17B). There was a significant (P<0.05, P<0.01, or even P<0.001) decrease of serum LDL-c level in each Beinaglutide treatment group compared with that in the control group (FIG. 17F), while no significant effect was observed for serum TBIL (FIG. 17C) and TG levels (P>0.05) (FIG. 17D). The results indicate that Beinaglutide significantly decreased some liver injury parameters, and it also significantly decreased serum total cholesterol and LDL-c levels. Thus, Beinaglutide may have the effects on lowering blood lipid and alleviating liver injuries caused by NASH.

The right middle lobes of livers were collected for TC, TG contents detection. No statistically significant effect on lowering liver TC content was seen in Beinaglutide treatment groups when compared with that in the control group (FIG. 18A). However, liver TG contents were dramatically decreased in Beinaglutide 1.2 mg/kg and 2.4 mg/kg treatment groups (FIG. 18B). These data suggest that Beinaglutide can significantly decrease liver fat content, and may have therapeutic effects on steatosis induced by NASH.

Effects on Liver Fibrosis

The right middle lobe of each liver was collected and total RNA was extracted from liver tissue at the end of study. The mRNA expression levels of fibrosis related genes, Col1a1, Tgfb1 and Acta2, were evaluated by qRT-PCR.

The relative mRNA expression level of Col1a1 was significantly decreased in each Beinaglutide dose group compared with the control group, while no statistically significant differences were observed for Tgfb1 and Acta2 gene expression levels (FIGS. 19A-19C). These data indicate that Beinaglutide can inhibit Col1a1 gene expression and Type I collagen formation, thereby to reduce liver fibrosis of HFD-NASH mice.

The left lateral lobe of each liver was collected and H&E staining, Sirius red staining and α-SMA IHC staining were performed to examine liver fibrosis severity. Staining positive areas were analyzed under double blind mode or by software automatic calculation.

H&E staining results showed that Beinaglutide 0.6 mg/kg and 2.4 mg/kg treatment significantly (P<0.05) decreased liver fibrosis scores when compared with the control group (FIGS. 20A-20C). Sirius red staining results show that the percentage of staining positive area appeared to have decreased in each Beinaglutide treatment group when compared with that of the control group (FIGS. 21A-21C).

The liver fibrosis pattern scoring results show that the number of animals with significant fibrosis severity was reduced after Beinaglutide 2.4 mg/kg treatment, suggesting that Beinaglutide can ameliorate pathological indication of NASH fibrosis (FIG. 21C). α-SMA IHC staining results show that α-SMA positive areas decreased. In all Beinaglutide treatment groups (FIGS. 22A-22B), suggesting that Beinaglutide can reduce the activation of hepatic stellate cells and reduce the potentiality of tissue fibrosis.

Effects on Liver Histopathology Injuries in HFD-NASH Mice

After 11 weeks of treatment at the end of the in vivo study, the left lateral lobe of each liver was collected, and H&E or oil red staining were performed. Double-blinded scoring on H&E stained slides and software automatic evaluation on oil red stained slides were carried out.

H&E staining results show that the area of liver macrovesicular steatosis was significantly decreased in Beinaglutide 2.4 mg/kg treatment group when compared with that in the control group (FIG. 23B). The NAS score in every Beinaglutide treatment group appeared to have decreased (FIG. 23A).

The animal number of each NASH severity grade was calculated based on NASH scoring criteria. For indexes of steatosis, steatosis location, interlobular inflammation, the animal number with severe pathology decreased after Beinaglutide 2.4 mg/kg treatment (FIGS. 24A-24E), indicating that the Beinaglutide administration can improve some of the clinical histopathological features of NASH.

In this study, the lipid droplets positions shifted after Oil red staining, so these results were not used as the basis for efficacy analysis.

In sum, the histopathological results show that 11 weeks of Beinaglutide treatment significantly decreased liver fibrosis score, macrovesicular lipid droplets area, and ameliorated steatosis, steatosis location and interlobular inflammation in comparison with those of the control group, indicating that Beinaglutide can significantly improve liver fibrosis injury observed in NASH model, and to certain degree, improve liver steatosis and interlobular inflammation.

Accordingly, an HFD-induced NASH model was established using ob/ob mice. The results revealed that after HFD induction for 17 weeks, the HFD-NASH mice showed significant body weight gain, food consumption reduction and NASH characteristics, including increased liver weight and liver index, elevated serum liver function injury parameters, higher serum lipids and liver TC/TG contents, augmented liver fibrosis related gene expression. Meanwhile, histopathological examination revealed that inflammatory cells infiltration, steatosis, ballooning degeneration, liver fat deposition, fibrin deposition and liver fibrosis severity were significantly increased in the liver tissues of the HFD-NASH mice. Therefore, the liver injury characteristics of mice. In the HFD-NASH model were highly similar to those observed in clinical NASH, and the pathogenesis was similar to clinical NASH accompanied by obesity, insulin-resistant diabetes and hyperlipidemia. Therefore, the HFD-NASH model is a successful animal model of NASH and can be used to evaluate the pharmacodynamics of the therapeutic drugs for NASH.

After Beinaglutide treatment for 11 weeks, body weight, body weight change percentage, liver weight and liver index significantly decreased in Beinaglutide 0.6 mg/kg, 1.2 mg/kg, and 2.4 mg/kg treatment groups when compared with those in the control group, suggesting that Beinaglutide administration can slow down body weight gain, liver weight and liver index increase of the HFD-NASH mice. Average food consumption in Beinaglutide 0.6 mg/kg, 1.2 mg/kg, 2.4 mg/kg treatment groups were lower than that in the control group, indicating that Beinaglutide treatment can inhibit appetite. Therefore, reducing energy intake from the source may have, to certain degree, therapeutic effects on controlling body weight gain, lowering obesity related risks and ameliorating liver injury caused by free fatty acids (FFA).

As for serum biochemical indexes for liver function, serum and liver lipid contents, and gene expression in liver tissues, the results showed that after Beinaglutide treatment for 11 weeks, the serum ALT, TC levels and liver TG content significantly reduced in Beinaglutide 1.2 mg/kg and 2.4 mg/kg groups. Also, the serum LDL-c levels and liver Col1a1 gene expression levels were significantly decreased in Beinaglutide 0.6 mg/kg, 1.2 mg/kg and 2.4 mg/kg treatment groups, suggesting that Beinaglutide can reduce blood lipids, decrease liver fat content, inhibit type I collagen production, and thus alleviate liver injury of the mice with HFD-NASH.

Additionally, histopathological results showed that the liver fibrosis scores significantly decreased in Beinaglutide 0.6 mg/kg and 2.4 mg/kg treatment groups compared to that in the control group at the end of study. The area of macrovesicular fat droplets in liver tissue significantly reduced in Beinaglutide 2.4 mg/kg treatment group; and the NAS scores appeared to have decreased by Beinaglutide 0.6 mg/kg, 1.2 mg/kg and 2.4 mg/kg treatments. For steatosis, steatosis location, interlobular inflammation, liver fibrosis grades and pattern, the numbers of animals with severe grading were decreased after Beinaglutide 2.4 mg/kg treatment. The results suggested that Beinaglutide administration can also improve some of the histopathological changes of the mice with NASH.

Therefore, Beinaglutide demonstrated therapeutic effects in HFD-NASH mice, especially in reducing body weight gain and liver weight/index, lowering blood lipids and visceral fat content, decreasing liver fibrosis gene expression levels and improving liver histopathological changes. Accordingly, under the test conditions of this study, Beinaglutide ameliorated NASH at different stages of the disease development.

1. A method of treating or preventing non-alcoholic steatohepatitis (NASH) in a subject comprising administering a composition comprising an effective amount of one or more first active ingredients selected from the group consisting of GLP-1 and GLP-1 analogs to the subject to treat or prevent NASH.

2. The method of claim 1, wherein the subject has NASH or has an elevated risk of having NASH.

3. The method of claim 1, wherein the GLP-1 analogs are selected from the group consisting of GLP-1 (7-37), GLP-1 (7-36), and GLP-1 (7-35).

4. The method of claim 1, wherein the GLP-1 and/or GLP-1 analogs have a C-terminal free carboxyl group.

5. The method of claim 1, wherein the GLP-1 analog is Beinaglutide.

6. The method of claim 1, wherein the GLP-1 and/or GLP-1 analogs are administered in a range from about 0.00070 mg/kg to about 0.0197 mg/kg body weight.

7. The method of claim 1, wherein the composition is administered once a day, twice a day, three times a day, or four times a day.

8. The method of claim 1, wherein the GLP-1 and/or GLP-1 analog(s) is administered at a daily dosing of about 40 μg to about 14,000 μg, about 40 μg to about 13,500 μg, about 50 μg to about 14,000 μg, about 50 μg to about 13,500 μg, about 40 μg to about 12,030 μg, about 50 μg to about 12,040 μg, about 2,010 μg to about 14,000 μg, about 1,510 μg to about 13,500 μg, about 250 μg to about 6,000 μg, about 250 μg to about 5,700 μg, about 300 μg to about 6,000 μg, about 300 μg to about 5,700 μg, about 480 μg to about 700 μg, about 480 μg to about 600 μg, about 540 μg to about 700 μg, or about 540 μg to about 600 μg.

9. A pharmaceutical composition comprising an effective amount of one or more first active ingredients selected from the group consisting of GLP-1 and GLP-1 analogs for treating or preventing NASH in a subject.

10. The pharmaceutical composition of claim 9, wherein the subject has NASH or has an elevated risk of having NASH.

11. The pharmaceutical composition of claim 9, wherein the GLP-1 analogs are selected from the group consisting of GLP-1 (7-37), GLP-1 (7-36), and GLP-1 (7-35).

12. The pharmaceutical composition of claim 9, wherein the GLP-1 and/or GLP-1 analogs have a C-terminal free carboxyl group.

13. The pharmaceutical composition of claim 9, wherein the GLP-1 analog is Beinaglutide.

14. The pharmaceutical composition of claim 9, wherein the GLP-1 and/or GLP-1 analogs are present at a concentration of 2 mg/mL.

15. The pharmaceutical composition of claim 9, wherein the pharmaceutical composition is preloaded into an administration device.

16. The pharmaceutical composition of claim 15, wherein the administration device is an injection pen or a pump.

17. The pharmaceutical composition of claim 9, further comprising one or more pharmaceutically acceptable excipients.

18. The pharmaceutical composition of claim 9, wherein the pharmaceutical composition is formulated for subcutaneous injection, intraperitoneal injection, intravenous injection, or infusion.
